(19) United States
(12) Patent Application Publication
(10) Pub. No.: US 2004/0053225 A1
(43) Pub. Date: Mar. 18, 2004

(54) Enhancement of virus induced gene silencing (VIGS) through viral-based expression of inverted-repeats

(75) Inventors: Gregory P. Pogue, Vacaville, CA (US); Christophe Lacomme, Scotland (GB)

Correspondence Address: LARGE SCALE BIOLOGY CORPORATION, 3333 VACA VALLEY PARKWAY, SUITE 1000, VACAVILLE, CA, 95688, US

(73) Assignee: LARGE SCALE BIOLOGY CORPORATION, Vacaville, CA

(21) Appl. No.: 10/660,860

(22) Filed: Sep. 12, 2003

Related U.S. Application Data
(60) Provisional application No. 60/410,879, filed on Sep. 13, 2002.

(51) Int. Cl. C12Q 1/70; A01K 67/00; C07H 21/04; A01H 1/00

(52) U.S. Cl. 435/5; 800/8; 800/280; 536/23.72

(57) ABSTRACT

Virus-based expression vectors carrying sequences corresponding to endogenous host genes trigger silencing through a homology-dependent RNA degradation mechanism. Virus-induced gene silencing (VIGS), is useful as a reverse-genetic tool for use in functional genomic programs for loss-of-function transient assays-based screening. Described herein is an approach to enhance the robustness of the VIGS phenotype by increasing the level of dsRNA molecule production.

### PRIORITY CLAIM

This application claims benefit of the filing date of U.S. Provisional Application No. 60/410,879 filed Sep. 13, 2002.

### BACKGROUND

The infection of plants by many viruses induces an RNA-mediated defence response that targets the viral genome, and any foreign sequences inserted into it, in a sequence-specific manner. This phenomenon, termed virus induced gene silencing (VIGS, for a recent review see Voinnet, 2001), is related to post-transcriptional gene silencing (PTGS), a phenomenon referred to as co-suppression or RNAinterference (RNAi) in others systems (Cogoni and Macino, 2000). PTGS can lead to the degradation of homologous RNA from an endogenous gene or transgenes, as well as viral RNA (Fagart and Vaucheret, 2000).

The involvement of aberrant, double-stranded (ds)RNA molecules in initiating gene silencing was first revealed by analysis of transgenic loci in plants undergoing PTGS. Silencing was often correlated with the occurrence of aberrant tandem DNA insertions (Hamilton et al., 1998). Since then, such arrangements have been intentionally engineered to generate trangenics lines with an increased probability of undergoing PTGS (Smith et al., 2000; Varsha Wesley et al, 2001).

The role of dsRNA in initiating a silencing signal that generates a systemic VIGS response (reviewed by Voinnet 2001), or acting as a systemic signal itself (Voinnet et al., 2000; Mallory et al., 2001; Boutla et al, 2002) still remain to be determined. According to current models, following an initiation step involving dsRNA production and its subsequent processing into 21 -23nt RNA (short interfering RNAs, siRNAs); the antisense siRNAs, homologous to the targeted transcripts, are subsequently incorporated into the RNA-induced silencing complex (RISC), a multi-component ribonuclease, to provide homology-based RNA degradation (Voinnet, 2001; Baulcombe, 2001).

It is commonly believed that during replication of cytoplasmic, positive single-stranded (ss)RNA viruses, the replicative-form and rcplicative-intermediates may represent the pool of dsRNAs that trigger VIGS initiation (Voinnet, 2001). In this model, negative ssRNA is used as a template by viral-encoded RNA-dependent RNA polymerase (RdRp) for generation of genomic and subgenomic positive ssRNA (Buck, 1999). This is corroborated by the fact that VIGS, unlike transgene-induced PTGS, is not affected in Arabidopsis mutants defective in RdRp and RNA helicase-like proteins (Dalmay et al., 2000), such activities being unnecessary because of corresponding virally encoded proteins.

The VIGS phenomenon can be used in transient knockout approaches for functional analyses of endogenous genes, as a consequence of homology-dependent RNA degradation mediated by a recombinant virus harbouring a host gene sequence (Baulcombe, 1999). So far, VIGS technology is restricted to the use of a few viruses in a limited number of hosts, mainly Nicotiana benthamiana and Arabidopsis thaliana (Voinnet, 2001). Recently, a monocot-infecting vector has been tested as a potential VIGS vector for cereals, broadening the potential use of VIGS to monocot species (Holzberg et al., 2002). Another problem with this approach to functional screening is the variable phenotype often associated with a VIGS response, limiting its use to a small number of viruses that are strong VIGS inducers, such as tobacco rattle virus (TRV; Ratcliff et al., 2001).

The development of VIGS as a more efficient genomic tool has involved the identification of suitable virus-host systems and the selection of viruses that trigger a stronger VIGS response (Ratcliff et al., 2001), or do not suppress PTGS (Voinnet et al., 1999). An unexplored route to enhance the effectiveness of VIGS vectors is to increase their ability to generate dsRNA.

Here we present a method that boosts the production of dsRNA through the engineering of inverted-repeats into viral-based expression vectors. We show that, in the case of cytoplasmically replicating viruses, such structures strongly enhance VIGS in different plant-virus systems.

Waterhouse literature, complementary sequences flanked by introns or hairpin RNAs delivered from the nucleus of plants.: e.g.

Stoutjesdijk et al., 2002. Plant Physiol. 129:1723-31

Beclin et al., 2002. Curr. Biol. 16:684-8.

Wang and Waterhouse 2002. Curr. Opin. Plant Biol. 5:146-50.

Wesley et al., 2001. Plant J. 27:581-90.

Wang et al., 2001. RNA 7:16-28.

### SUMMARY OF THE INVENTION

Virus-based expression vectors carrying sequences corresponding to endogenous host genes trigger silencing through a homology-dependent RNA degradation mechanism. This phenomenon called virus-induced gene silencing (VIGS), has the potential for development as a-powerful reverse-genetic tool for use in functional genomic programs for loss-of-function transient assays-based screening. Here we describe an approach to enhance the robustness of the VIGS phenotype by increasing the level of dsRNA molecule production, a critical step in the VIGS response. Expression from a plant viral vector of 40-60 bp direct inverted-repeats generates RNA molecules that form dsRNA hairpins. A tobacco mosaic virus (TMV)-based vector expressing such inverted-repeats corresponding to a green fluorescent protein (GFP) transgene or a phytoene desaturase (PDS) endogenous gene resulted in a higher number of silenced leaves exhibiting a stronger VIGS phenotype in comparison to constructs expressing corresponding cDNA fragments in a sense or antisense orientation. Real time PCR indicated that up to a three-fold decrease of target mRNA accumulation was detected in VIGSed tissues triggered by constructs expressing inverted-repeats than sense or antisense constructs. Moreover, an enhanced PDS VIGS phenotype was observed using a different virus, barley stripe mosaic virus, in barley plants. This is the first demonstration that suggests that dsRNA formation is a limiting factor in the VIGS phenomenon and that VIGS can be significantly improved through expression of small inverted-repeats and generate a more robust loss-of-function phenotype.

### BRIEF DESCRIPTION OF THE DRAWINGS

FIGS. 1A and 1B show a TMV-based expression of GFP direct inverted-repeats enhances VIGS of a GFP transgene. Specifically, FIG. 1A is a schematic representation (not to scale) of TMV expression vector genome organisation and derived constructs generated to express GFP cDNA fragment for VIGS. Right- and left-pointing arrows represent sense and antisense orientation of cDNA fragments, numbers indicate the nucleotide length and the unrelated spacer sequence is represented as an orange block. The predicted structures of RNA transcripts are presented on the right side. REP, replicase; HEL, helicase; MP, movement protein; GFP, green fluorescent protein; CP, coat protein. FIG. 1B shows VIGS kinetic of GFP transgene in Nicotiana benthamiana 35S-GFP plants observed under UV-light at 9 dpi (upper panels i-iv) and 12 dpi (lower panels v-viii) challenged with the following TMV.GFPs₂₀₀ (panels i and v), TMV.GFPs₄₀ (ii, vi) TMV.hpGFP₂₀₀-loop (iii, vii) and TMV.hpGFP₄₀ (iv, viii). Silenced areas appear in purple as opposed to bright green fluorescence. Inoculated leaves are pointed with a white arrow. This typical phenotype of GFP silenced plants is representative of at least 5 independent experiments.

FIGS. 2A and 2B show a TMV-based expression of PDS direct inverted-repeats enhances VIGS of endogenous PDS gene on a size-dependent manner. Specifically, FIG. 2A shows a schematic representation of expressed PDS fragment cDNA inserts as depicted in FIG. 1A. FIG. 2B shows a phenotype of N. benthamiana plants undergoing VIGS of endogenous PDS gene at 12 dpi, challenged with TMV.PDSs₁₁₀, TMV.PDSas₁₁₀, TMV.hpPDS₄₀ and TMV.hpPDS₆₀ constructs. This typical phenotype of PDS silenced plants is representative of at least 5 independent experiments.

FIGS. 3A, 3B and 3C show RT-PCR and real time PCR of PDS and ubiquitin mRNA levels in silenced and non-silenced control N. benthamiana plants. FIG. 3A is a schematic representation of N. benthamiana PDS cDNA with the position of the primers and size (not to scale) of amplification products used for RT-PCR and real time PCR (Taqman). Boxed arrows represent the location, size (not to scale) and orientation of inserted cDNA fragments used for triggering VIGS. FIG. 3B shows RT-PCR of N. benthamiana PDS silenced and control plants in response to challenge with TMV constructs. Both RT-PCR products corresponding to endogenous PDS mRNA (690 bp) and ubiquitin (250 bp) have been assessed. PCR conditions ranging from 20 to 50 amplification cycles were tested in both cases. Presented here are PCR conditions of 30 cycles corresponding to the Log-linear phase of amplified PDS PCR product in non-silenced tissues. NTC: non-template control, P1: plant 1, P2: plant 2, P3: plant3. FIG. 3C shows a histogram representation of the percentage of relative expression levels of normalised PDS mRNA in silenced leaf tissues to TMV-challenged control plants.

FIGS. 4A, 4B, and 4C show inverted-repeat expression enhances VIGS triggered by barley stripe mosaic virus-derived vector in barley plants. FIG. 4A is a schematic representation of barley stripe mosaic virus genome organisation and nature of expressed PDS fragment. Genomic organisation of BSMV RNAs α, β and γ as described by Holzberg et al. (2002). RNA β is a βa (CP)-deletion mutant. Inserted PDS fragments, orientation and size (not to scale) are as a described previously. Origins of PDS cDNA fragments in inverted-repeat are Hv for Hordeum vulgare and Nb for Nicotiana benthamiana. FIG. 4B shows a sequence alignment of 60-bp barley and N. benthamiana PDS fragments used to generate inverted-repeats to be expressed in BSMV. Conserved nucleotides are in red, the boxes delineate the largest sequence identity stretches of 8 and 14 nucleotides between the two PDS cDNAs. FIG. 4C shows a phenotype of barley leaves (third-upper uninoculated) from 3 independent plants undergoing VIGS of PDS. Pictures have been taken at 14 dpi post-challenge by BSMV.PDSs₁₈₀, BSMV.PDSas₁₈₀, BSMV.hpPDS_{Hv60} and BSMV.hpPDS_{Nb60} constructs. These results are representative of at least 5 independent experiments.

FIGS. 5A and 5B show RT-PCR and real time PCR of PDS and ubiquitin mRNA levels in silenced and non-silenced control barley plants. FIG. 5A is aschematic representation of barley PDS cDNA with the position of the primers and size (not to scale) of amplification products used for real time PCR (Taqman) represented above. Below are boxed arrows representing the relative position, size (not to scale) and orientation of inserted cDNA fragments from barley and N. benthamiana PDS cDNA cloned into the BSMV-based vector used for triggering VIGS. The predicted full-length of the truncated barley PDS cDNA is represented in a dotted rectangle using N. benthanziana as a reference. FIG. 5B is a histogram representation of the percentage of relative expression levels of normalised PDS mRNA in silenced leaf tissues to BSMV.GFP-challenged control plants.

### DETAILED DESCRIPTION

### EXAMPLE 1

Cloning of Inverted-Repeats Into TMV Vector and Infectivity of Constructs

TMV-based expression vectors were generated in order to compare VIGS of a GFP transgene, expressed constitutively in N. benthamiana. VIGS was induced by a single RNA fragment, or its corresponding hairpin generated as a consequence of direct inverted-repeat expression.

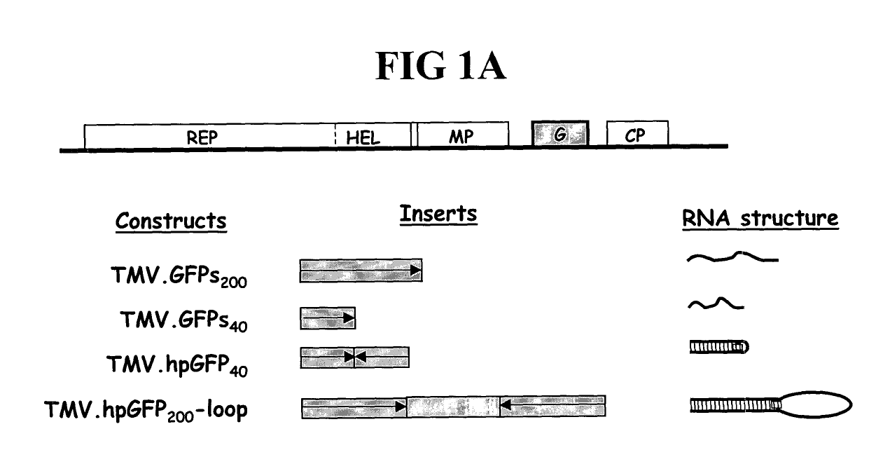

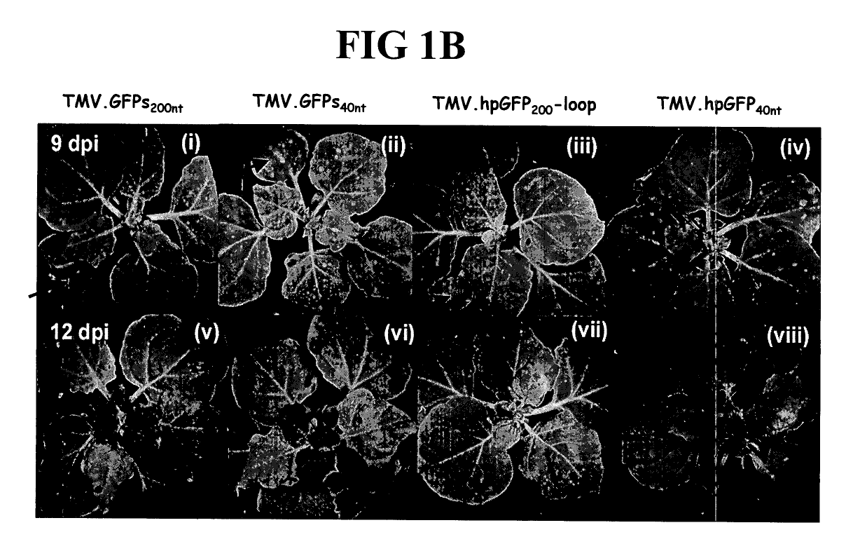

GFP fragments of 40 bp and 200 bp were cloned in sense orientation into a TMV vector to produce, respectively, constructs TMV.GFPs₄₀ and TMV.GFPs₂₀₀ (FIG. 1A).

The 40 bp sequence was also inserted into the TMV vector as a 40 bp direct inverted-repeat to produce TMV.hpGFP₄₀ (FIG. 1A). Attempts to produce similar constructs with 100 bp and 200 bp direct inverted-repeats were unsuccessful, suggesting a possible strong selection against 100 bp- and 200 bp-inverted-repeats sequences being propagated in E. coli cells. To obtain inverted-repeats of GFP fragments up to 200 bp in length into TMV, a three-step cloning procedure was used in which a non-palindromic unrelated spacer fragment was introduced between the two inverted-repeats. Following transcription, this resulted in the generation of an imperfect dsRNA hairpin harbouring a loop structure (FIG. 1 a, construct TMV.hpGFP₂₀₀-loop). In-vitro assembled TMV virions, generated by incubation of in-vitro transcripted RNA with TMV-coat protein (CP) monomers, produced modal length TMV rods for all constructs engineered (data not shown), indicating that the inserted sequences did not prevent assembly of the viral particles. Infectivity tests were carried out on a resistant host (Nicotiana glutinosa, genotype NN), which produced localised necrotic spots, characteristic of N-gene mediated hypersensitive response when challenged with TMV.GFPs₄₀, TMV.GFPs₂₀₀ and TMV.hpGFP₄₀ constructs. In contrast to the others constructs, smaller necrotic lesions were eventually observed in response to challenge with TMV.hpGFP₂₀₀-loop and TMV.hpGFP₁₀₀-loop constructs (data not shown). Viral accumulation, assessed by western blotting of TMV.CP accumulation, indicated low accumulation levels for TMV.hpGFP₂₀₀-loop and TMV.hpGFP₁₀₀-loop in comparison with TMV.GFPs₄₀, TMV.GFPs₂₀₀ and TMV.hpGFP₄₀ constructs, suggesting poor infectivity and accumulation of constructs harbouring larger inverted-repeats.

### EXAMPLE 2

TMV-Based Expression of GFP 40 bp Inverted-Repeats is a Strong VIGS Inducer of a GFP Transgene

The ability of the above constructs to trigger VIGS of a transgene was assessed on N. benthamiana plants expressing GFP. By 9 days post-inoculation (dpi), TMV.GFPs₄₀ and TMV.GFPs₂₀₀ caused the appearance of silenced areas on the leaf, observed as small purple spots under UV-light observation (FIG. 1b panel i and ii), a characteristic of the loss of GFP fluorescence on sink leaves (Ruiz et al., 1998). By 12 dpi GFP silencing by these two constructs was observed on complete young expanding leaves (FIG. 1B TMV.GFPs₄₀ panel v, and TMV.GFPs₂₀₀ panel vi). Not until 12 dpi, did TMV.hpGFP₂₀₀-loop induce a silencing phenotype observed as purple areas in close vicinity to veins on sink leaves (FIG. 1B panel vii), and this was weaker than the VIGS induced by TMV.GFPs₄₀ and TMV.GFPs₂₀₀.

In contrast, by 9 dpi in plants challenged with TMV.hpGFP₄₀ (FIG. 1B, panel iv), development of numerous purple areas were observed in most of the upper uninoculated leaves. By 12 dpi the silenced area covered not only sink tissues, where the youngest expanding leaves appeared purple, but also upper non-inoculated source leaves (FIG. 1B, panel viii). In some instances, almost complete silencing was observed throughout the plant (FIG. 1B, panel viii).

### EXAMPLE 3

TMV-Based Expression of 40 bp- and 60 bp-Inverted-Repeats Strongly Enhances VIGS of an Endogenous PDS Gene

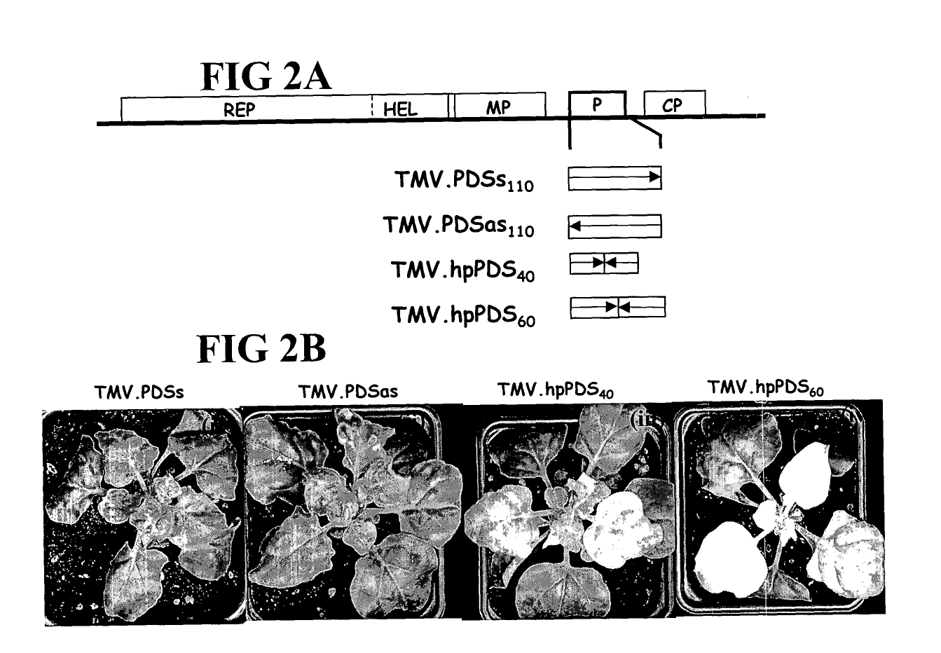

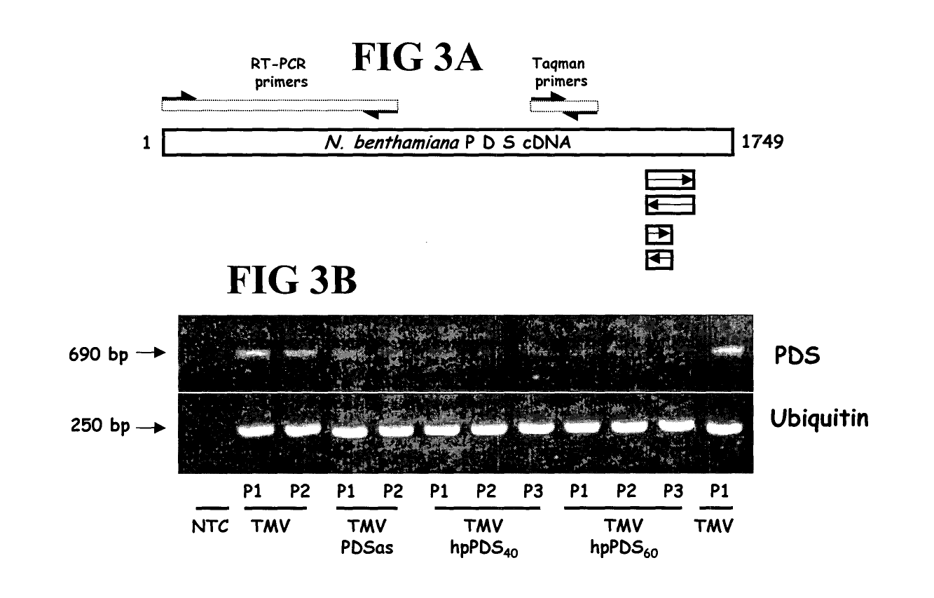

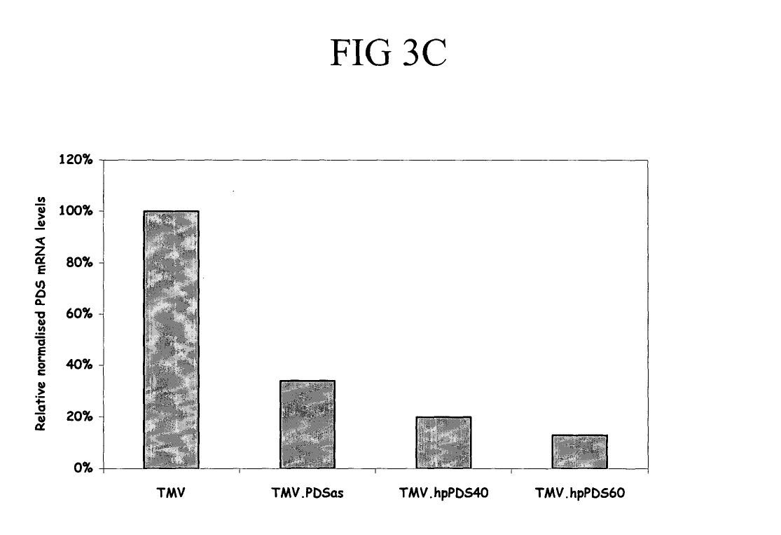

To evaluate the hairpin-based VIGS-enhancement of an endogenous gene rather than a transgene, the hairpin-based silencing of the endogenous phytoene desaturase (PDS) gene was tested. VIGS of PDS has been well characterised (Kumagai et al., 1995; Ratcliff et al., 2001) and results in a discemable white phenotype due to photo-bleaching that arises as a consequence of chlorophyll pigment degradation (Kumagai et al, 1995). TMV vectors harbouring a 110 bp PDS cDNA fragment encompassing nucleotides 1490-1600 from N. benthamiana PDS cDNA (FIG. 3B), in sense or antisense orientation (FIG. 2A, TMV.PDSs₁₁₀ and TMV.PDSas₁₁₀), and direct inverted-repeats of 40 bp and 60 bp (FIG. 2A, TMV.hpPDS₄₀, TMV.hpPDS₆₀) were engineered. For TMV.PDSas₁₁₀ construct photo-bleaching occurred by 12 dpi and was visible only in a few discrete areas as white patches adjacent to veins in sink leaves (FIG. 2B, panel ii). The TMV.PDSs₁₁₀ construct had triggered even weaker photo-bleaching, solely in the vicinity of the veins in the youngest leaves (FIG. 2B, panel i). In contrast, at this time point plants challenged with constructs TMV.hpPDS₄₀ and TMV.hpPDS₆₀, showed photo-bleaching over entire sink leaves and upper uninoculated source leaves (FIG. 2B, panels iii and iv), with stronger photo-bleaching being observed in TMV.hpPDS₆₀ challenged plants than TMV.hpPDS₄₀ challenged plants (FIG. 2B, panel iv and iii).

The effect of hairpin production on RNA degradation was analysed at the molecular level by monitoring PDS mRNA accumulation levels in plants undergoing silencing in response to challenge by TMV.PDSas₁₁₀, TMV.hpPDS₄₀ and TMV.hpPDS₆₀ constructs. In the first instance, RT-PCR tests were carried out by sampling the silenced leaf with the highest proportion of white tissue at 12 dpi, using 2 to 3 different plants per construct. The PDS cDNA region, which did not overlap the region cloned into the TMV vectors to elicit VIGS (FIG. 3A), was shown to be amplified at lower levels in TMV.PDSas₁₁₀ silenced tissues than control TMV infected tissues (FIG. 3B). Even less PDS PCR product was amplified in leaves undergoing VIGS triggered by TMV.hpPDS₄₀ and TMV.hpPDS₆₀ constructs (FIG. 3B). The levels of control ubiquitin RT-PCR product were similar in all tissues tested (FIG. 3B). Real-Time PCR of silenced samples was then used to assess the amounts of PDS mRNA in all the samples relative to an internal control ubiquitin mRNA. In comparison to TMV challenged plants, PDS mRNA was shown to be reduced by 65% of control levels in TMV.PDSas₁₁₀ infected plants and between 80% to 90% of control levels in plant challenged with TMV.hpPDS₄₀ and TMV.hpPDS₆₀ (Table 1 and FIG. 3C). No statistical difference was determined in the mRNA levels detected in leaves undergoing silencing in response to TMV.hpPDS₄₀ and TMV.hpPDS₆₀ (Table 1), despite the fact that TMV.hpPDS₆₀ triggered a stronger photo-bleaching phenotype (FIG. 2B).

### EXAMPLE 4

Hairpin-Mediated VIGS Enhancement in Monocot Species

To determine whether expression of inverted-repeats could enhance VIGS in other systems, we tested a recently described VIGS monocot vector, barley stripe mosaic virus (BSMV, Holzberg et al., 2002), on barley to evaluate its capacity to enhance silencing of barley PDS gene.

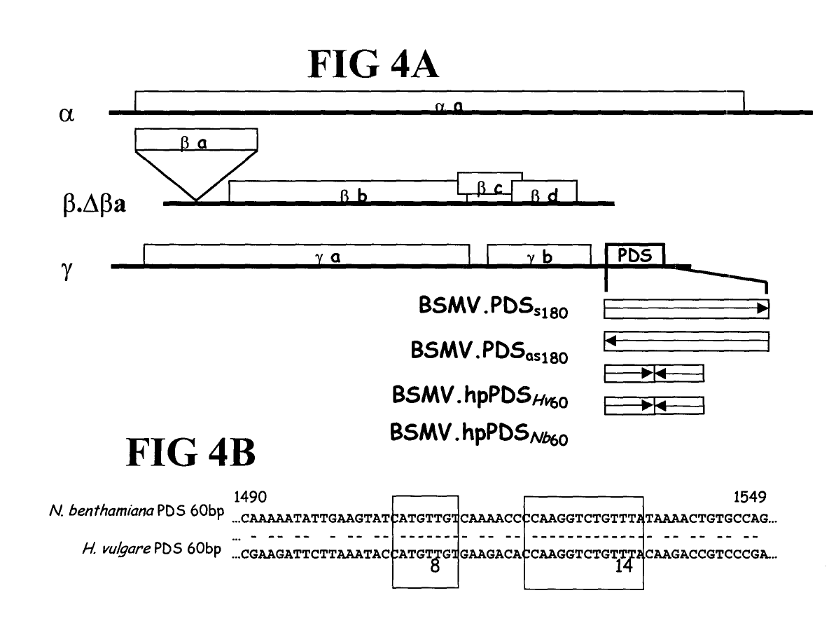

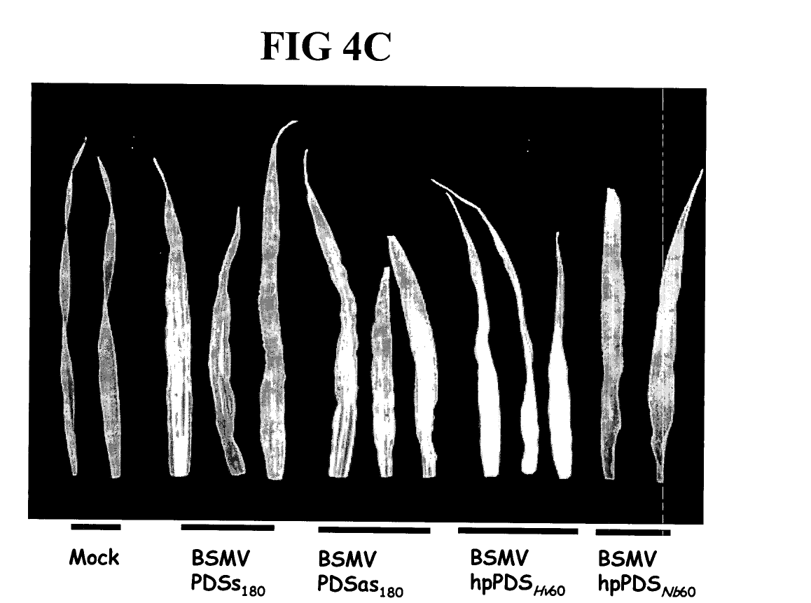

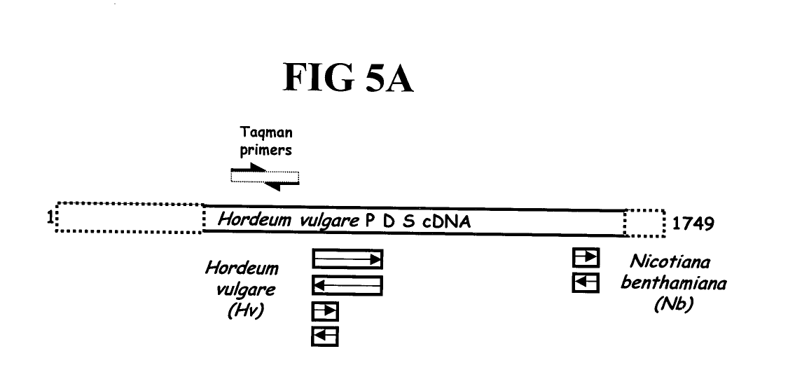

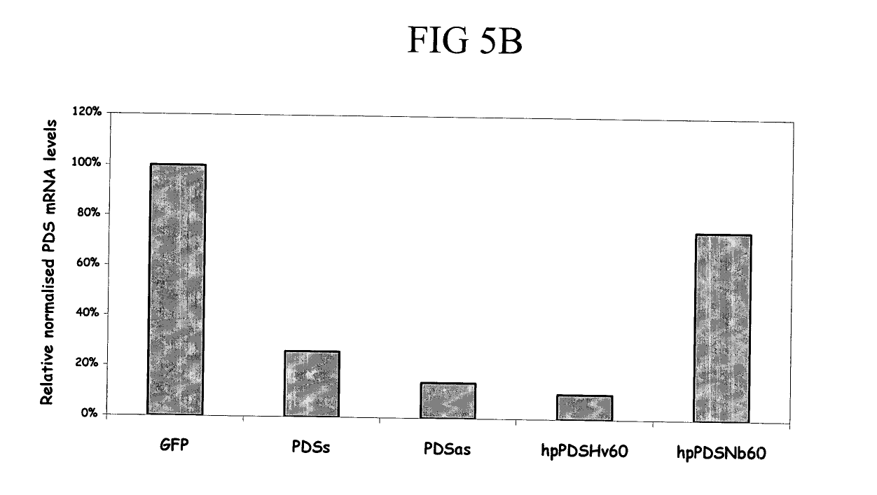

Similarly, 180-nucleotide fragments of barley PDS cDNA in sense and antisense orientation (Holzberg et al., 2002) and 60 bp-inverted-repeats of the same cDNA region were cloned into BSMV (FIG. 4A, constructs BSMV.PDSs₁₈₀, BSMV.PDSas₁₈₀, BSMV.hpPDS_{Hv60} and FIG. 5A). Since BSMV infection caused chlorosis and necrosis in the early stages of the infection process, a vector carrying the heterologous N. benthamiana 60-bp PDS inverted-repeat fragment (FIG. 4A, BSMV.hpPDS_{Nb60}) was used as a control to differentiate symptoms resulting from infection and PDS VIGS phenotype in barley plants. This segment corresponding to nucleotides 1490-1550 (FIG. 5A) was chosen because of the low nucleotide identity occurring in this region between the barley and N. benthamiana PDS sequences not exceeding a stretch of 14 nucleotides (FIG. 4B). FIG. 4C shows third upper uninoculated barley leaves at 14 days post-inoculation. These leaves have been chosen as they develop the strongest VIGS phenotype, commonly observed from 8 dpi, and do not show BSMV-dependent infection symptoms usually observed from 4 dpi on the inoculated leaf up to the second upper uninoculated leaves. For plants challenged with constructs BSMV.PDSs₁₈₀ and BSMV.PDSas₁₈₀, white streaks of photo-bleached tissue of variable width were observed by 14 dpi, however, green streaks of non-photo-bleached tissues remained (FIG. 4C). In contrast, for plants challenged with BSMV.hpPDS_{Nb60}, despite displaying comparable initial symptoms to other constructs, no obvious photo-bleaching was observed by 14 dpi. At this time point the plants challenged with BSMV.hpPDS_{Hv60} displayed the strongest photo-bleaching phenotype in comparison to BSMV.PDSs₁₈₀ and BSMV.PDSas₁₈₀ constructs, as the white photo-bleached area covered most of the leaf (FIG. 4C).

Both PDS and ubiquitin mRNA levels were assessed by real-time PCR in barley leaves infected with the above BSMV constructs by amplifying, as before, a PDS cDNA region, which did not overlap the region cloned into the BSMV vectors to elicit VIGS (FIG. 5A). The BSMV.hpPDS_{Nb60} construct harbouring the PDS fragment from N. benthamiana did not trigger any obvious photo-bleaching and no significant PDS mRNA decrease in comparison to BSMV.GFP control plants was detected (Table 2). PDS mRNA levels were shown to be reduced by 75-85% of control levels in BSMV.PDSs₁₈₀, BSMV.PDSas₁₈₀ and BSMV.hpPDS_{Hv60} silenced tissue (Table 2 and FIG. 5A). Although the apparent phenotypic differences, the determined PDS mRNA levels were not statistically different between VIGS tissue triggered by BSMV.PDSs₁₈₀, BSMV.PDSas₁₈₀ and BSMV.hpPDS_{Hv60} constructs (Table 2).

Discussion

We have shown that expression of direct, inverted-repeats that fold as dsRNA hairpin structures strongly enhance VIGS caused by a cytoplasmic virus. This effect was analysed by targeting of either a transgene or endogenous gene using two different plant virus-based expression systems. In both cases, hairpin-based expression from these virus vectors triggered a more robust VIGS event that occurred more rapidly and uniformly than that observed with sense or antisense constructs harbouring corresponding cDNA fragments. Our results indicate that the nature and the amount of dsRNA molecules are crucial during VIGS generated by cytoplasmic viruses, as is the case in transgene-mediated PTGS (Smith et al., 2000; Varsha Wesley et al., 2001). Moreover, our results show for the first time that the threshold level of dsRNA formation is influential in generating a VIGS response, suggesting that occurrence of dsRNA structures is a limiting step in the VIGS process.

VIGS initiation is believed to occur through dsRNA duplexes that form during replication of positive-strand RNA viruses by intermolecular pairing of positive and negative ssRNA. For a recombinant virus carrying a sense or antisense cDNA sequence to generate VIGS, the amount of dsRNA duplexes results from the pairing between positive and negative genomic-RNA strands and with the corresponding endogenous mRNA. During TMV replication, the ratio of negative to positive strand is about 100:1 (Kielland-Brandt, 1974). In the case of TMV-based expression of inverted-repeats, driven by a duplicated coat protein subgenomic promoter (Shivprasad et al, 1999), each genomic and subgenomic RNA molecule synthesized (the latter accumulating between 2- to 3-fold in comparison to genomic RNA) will harbour inverted-repeat sequences, therefore reaching in total an estimated 200- to 300-fold increase in the amount of RNA molecules potentially generating dsRNA structures, sufficient to confer a stronger VIGS phenotype.

Virus-based expression of such inverted-repeats, or palindromic sequences, in genomic and subgenomic RNAs could lead to dsRNA duplex formation via two mechanisms: intermolecular pairing mediated by hybridisation, possibly between each replicative intermediate strands RNAs molecules or more likely through intramolecular pairing by self-hybridization of each individual RNA molecule synthesized. Although enhancement of silencing has been observed through intermolecular pairing in transgenic plants (Waterhouse et al., 1998) or more recently in a transient RNAi system (Schweizer et al, 2000; Tenllado and Ruiz 2001), approaches aimed at producing dsRNA through intramolecular pairing by expression of single self-complementary RNA generate a more robust phenotype (Waterhouse et al., 1998). Direct comparison of intramolecular and intermolecular-based dsRNA formation has been addressed in another report (Chuang and Meyerowitz, 2000), and has revealed a stronger silencing event obtained as a result of expression of hairpin-loop structures compared to sense and antisense coexpression. Recently, intramolecular pairing has been used in transient assays to interfere more efficiently with co-expressed genes (Johansen and Carrington, 2001; Schweizer et al., 2000), transgenes (Johansen and Carrington, 2001), or stable expression to confer PTGS to corresponding endogenous genes (Waterhouse et al., 1998; Levin et al. 2000; De Buck et al, 2001, Vasha Wersley et al, 2001).

Another crucial factor in silencing efficiency is the nature of the inverted-repeat sequence to be expressed. Direct inverted-repeats generating a loopless dsRNA structure generate a stronger silencing response both in the VIGS or PTGS response. Here, we have demonstrate that virus-based expression of short, direct inverted-repeats of 40- and 60-nucleotide length are more efficient than inverted-repeats of 100- and 200-nucleotide length separated by an unrelated non-palindromic insert. This improvement probably occurs as a result of higher stability of short, loopless duplexes and/or may reflect the low likelihood of hairpin-loop structures folding properly. Our results are comparable to those obtained in transgenic plants by Smith et al (2000), where despite stabilisation of 120- to 700-nucleotide inverted-repeat sequences by insertion of a spacer fragment (Muskens et al., 2000), this significantly decreased PTGS as opposed to a spacer sequence corresponding to a spliceable intron that generated a loopless dsRNA structure following transcription and splicing, thus triggering a strong PTGS.

Virus-vectors have their own limitations in terms of size and nature of foreign sequences that can be accommodated (Lacomme et al., 2001). This reflects either packaging constraints or genomic instability of inserted large inverted-repeat sequences probably through nonhomologous recombination (Simon and Bujarski, 1994). Indeed the incidence of recombination by promoting RdRp pausing between sense and antisense heteroduplexes or internal hairpin structures has been shown to be proportional to the length of the inserted sequence, such structures constituting recombination hotspots (Simon and Bujarski, 1994; Nagy and Simon, 1997). These could account for the poor infectivity and low accumulation levels observed with such constructs (TMV.hpGFP₂₀₀-loop and TMV.hpGFP₁₀₀-loop, TMV.hpPDS₂₀₀-loop, data not shown), hence producing lower amount of dsRNA molecules, triggering in turn a poorer VIGS phenotype in comparison to constructs harbouring sense or antisense cDNA fragments. This limitation could account for the discrepancy between the VIGS and PTGS phenotypes generated by expression of large imperfect inverted-repeats from a plant virus (this study) and transgenics plants where more consistent PTGS was observed than with sense or antisense transgenes (Smith et al., 2000; Waterhouse et al., 1998; DeBuck et al., 2001).

It is noteworthy that a significantly stronger VIGS of PDS generated by TMV constructs expressing 60-nucleotide hairpins was observed in comparison to their 40-nucleotide counterparts. TMV.hpPDS₄₀ triggers a milder photo-bleaching than TMV.hpPDS₆₀, suggesting that downstream VIGS events following dsRNA production were not saturated even when 40-nucleotide hairpins were expressed. This suggests that hairpin-sequence length is probably still a limiting factor in generating siRNAs to prime a RISC-mediated degradation step. If this is the case, it will be of importance to assess the ratio of target sequence-length to spacer sequence-length most compatible with cloning, in vivo propagation and efficient folding into dsRNA structures to potentate VIGS phenotype.

TMV-based expression of hairpin structures triggers a stronger RNA-mediated degradation of the endogenous PDS mRNA in comparison with constructs expressing the cDNA fragment in antisense orientation. Hairpin expression produced up to a 3-fold decrease of mRNA levels in comparison to the antisense construct. By 12 dpi, this 80% to 90% decrease in PDS mRNA levels is comparable to those obtained with PVX or TRV VIGS vectors at 21 dpi (Ratcliff et al., 2001). A higher proportion of photo-bleached tissue was observed with TMV.hpPDS₆₀ than TMV.hpPDS₄₀, however no statistically significant difference in determined transcript levels was observed. These data are consistent with previous findings that PDS mRNA levels do not always correlate with the degree of photo-bleaching (Ratcliff et al., 2001). Further, as observed with TMV.PDSas₁₁₀, significant PDS mRNA reductions from 60% to 65% did not result in strong photo-bleaching. It is probable that strong photo-bleaching occurs only when PDS mRNA levels decrease below those measured in VIGSed tissues induced by the antisense construct. On the other hand, below this level, it is likely that even marginal decreases in PDS mRNA levels will cause a stronger photo-bleached phenotype.

VIGS enhancement through hairpin expression occurred not only from a TMV vector but also from barley stripe mosaic virus (BSMV) on barley, suggesting that expression of such inverted-repeats trigger a similar effect in monocot species, and that in planta production of dsRNA is not hampered in BSMV-based vector constructs. For BSMV constructs, a slight PDS mRNA decrease resulted in a stronger VIGS phenotype when expressing barley PDS inverted-repeat in comparison to sense and antisense constructs, as previously observed with TMV constructs. The dsRNA formation of heterologous cDNA fragments generated by expression of N. benthamiana 60-nucleotide PDS cDNA inverted-repeat was not sufficient to trigger VIGS of the endogenous PDS barley gene. The two PDS cDNAs, despite displaying an overall 73% sequence identity between the selected 60-bp sequences, have a maximal 14-nucleotide stretch of identity. This result confirms the importance of sequence stretch identity length in VIGS initiation. As previously reported, the VIGS response necessitates a minimum sequence requirement of at least 23-nucleotide identity (Thomas et al., 2001).

The nature of VIGS inducers and their ability to enhance silencing is of prime importance in order to improve current VIGS system for generating more robust loss-of-function transient assay systems in plants. The fact that virus-based expression of hairpins results in VIGS enhancement in both dicot and monocot species, where for the former stable reverse-genetics approaches are not yet routine, strengthen its use in generating more robust loss-of-function assays, and in providing opportunities to increase the silencing capabilities of other VIGS vectors on a range of hosts.

Experimental Procedures

Construction of TMV-Derived Vectors

The TMV vector used in this study has been previously described (Lacomme and Santa Cruz, 1999); GFP and PDS cDNA fragments were inserted between the PacI and XhoI sites. The PacI site was used for the cloning of GFP and PDS inverted-repeats.

GFP Constructs

PCR fragments of 43 bp and 200 bp corresponding to nucleotides 332-375 and 332-532 of a GFP cDNA respectively (GenBank accession number U87625, Haseloff et al, 1997) were amplified from a GFP cDNA template using specific oligonucleotide primers incorporating restriction enzyme recognition sites at the 5′-(PacI) and 3′-(XhoI) termini for cloning into TMV to generate constructs TMV.GFPs₄₀ and TMV.GFPs₂₀₀ respectively. Subcloning of the 40 bp- direct inverted-repeat into TMV was achieved by subsequent XhoI digestion of the 40 bp- GFP PCR fragment followed by self-ligation, gel purification of the 80 bp concatenate fragment and PacI-digestion prior to cloning into a TMV vector (construct TMV.hpGFP₄₀). Construct TMV.hpGFP₂₀₀-loop was achieved by a three-step ligation of the 200 bp-GFP PCR fragment PacI-HindIII in the sense orientation to the HindIII-SpeI PCR fragment intron 2 of ST-LS1 gene (Eckes et al., 1986; Ibrahim et al., 2001; GenBank accession number X04753) and the SpeI-XhoI 200 bp-GFP PCR fragment in the antisense orientation. The resulting fragment was digested with PacI-XhoI and subcloned into TMV.

PDS Constructs

A 110 bp-cDNA fragment corresponding to nucleotides 1490-1600 from N. benthamiana PDS cDNA (GenBank accession number I23875) was PCR amplified using specific oligonucleotide primers incorporating restriction enzyme recognition sites at the 5′-(PacI) and 3′-(XhoI) termini for sense, or 5′-(XhoI) and 3′-(PacI) termini for antisense orientation. For cloning of direct inverted-repeats, 40 bp and 60 bp PCR fragments corresponding respectively to nucleotides 1490-1530 and 1490-1550 of the PDS cDNA were generated using oligonucleotide primers incorporating a PacI and an EcoRI site at their 5′- and 3′-termini respectively. Subsequent purification and EcoRI digestion of 40 bp and 60 bp PCR products was followed by self-ligation, PacI digestion and purification of the 80 bp and 120 bp inverted repeats to be inserted into the PacI site of a TMV vector.

Construction of BSMV Derived Vectors

The γRNA-based BSMV vector used in this study has been previously described (Holzberg et al., 2002) carrying sense and antisense orientations of a 180 bp-cDNA fragment from barley PDS cDNA (respectively constructs pγ.PDSs₁₈₀ and pγ.PDSas₁₈₀ further referred to as BSMV.PDSs₁₈₀ and BSMV.PDSas₁₈₀). For homogeneity of PDS sequences used as a silencing trigger, barley PDS hairpin construct (BSMV.hpPDS_{Hv60}) were derived from a 67 bp PCR fragment internal to the 180 bp PDS cDNA (GenBank accession number AY062039) fragment used for sense and antisense constructs. For cloning of direct inverted-repeats, a PacI-EcoRI fragment of 67 bp was PCR amplified using the following oligonucleotides primer combinations (5′-TAA ATT AAT TAA TTC CTG ATC GAG TCA AC-3′ (Seq ID No. 1) and 5′-ATT TGA ATT CTT TAT GAA ATT GAG-3′ (Seq ID No.: 2)). Subsequent purification and EcoRI digestion of the PCR product was followed by self-ligation, blunting and gel purification of the generated 118 bp fragment prior insertion into the blunt-ended PacI-NotI restriction sites from the pγ.bPDS4 vector (Holzberg et al., 2002) to generate BSMV.hpPDS_{Hv60} construct. BSMV.hpPDS_{Nb60} construct was obtained by blunt-end subcloning of the previously described 60-bp inverted-repeat from N. benthamiana PDS cDNA into the same PacI-NotI-digested and blunt-ended pγ.bPDS4 vector.

RNA Extraction and cDNA Synthesis

Total RNA was extracted from frozen silenced leaves using the Qiagen RNeasy plant mini kit (Qiagen Ltd), following the protocol supplied by the manufacturer. Prior to cDNA synthesis, RNA samples were treated with RNase-free DnaseI (DNA-free™ kit, Ambion Ltd), following the manufacturer recommendations. 5 μg of total RNA were used for first strand cDNA synthesis by oligodT priming using SuperScript™ II RNase H⁻ Reverse Transcriptase (Invitrogen Life Technologies Ltd) following manufacturer's recommendations.

RT-PCR and SYBR Real-Time RT-PCR Experiments

For RT-PCR analysis, primers that anneal outside the region of the PDS cDNA targeted for silencing were used to ensure that the endogenous gene is tested as indicated in FIG. 3A. Ubiquitin cDNA was used as an internal constitutively expressed control. First strand cDNA was used as template for PCR amplification through 25, 30, 35, 40, 45 and 50 cycles. As 30-cycles of amplification corresponded to the Log-linear phase of PDS PCR product amplification in the non-silenced control samples (data not shown), these conditions were selected for comparison of relative accumulation of both PDS and ubiquitin mRNAs in all samples.

Similarly, for SYBR real-time RT-PCR experiments, primer pairs were designed outside the region of the Nicotiana benthamiana and barley PDS cDNA targeted for silencing (FIGS. 3A and 5A) and for the internal control ubiquitin cDNA from both species using the Primer Express software supplied with the ABI PRISM 7700 Sequence Detection System (Applied Biosystems, USA) following the manufacturer's guideline for primer design. The following PDS primers were used: NtPDSfwd (5′-TTC TTC AGG AGA AAC ATG GTT CAA-3′ (Seq ID No.: 3)), NtPDSrev (5′-TCC ACA ATC GGC ATG CAA-3′ (Seq ID No.: 4)), NtUBIfwd (5′-TCC AGG ACA AGG AGG GTA TCC-3′ (Seq ID No. 5)), NtUBIrev (5′-TAG TCA GCC AAG GTC CTT CCA T-3′ (Seq ID No.: 6)), HvPDSfwd (5′-TGG AGC TTA TCC CAA TGT ACA GAA-3′ (Seq ID No.: 7), HvPDSrev (5′-TGT ATT CCC CTG GCT TGT TTG-3′ (Seq ID No.: 8)), HvUBIfwd (5′-GCA AGT AAG TGC CTG GTC ATG A-3′ (Seq ID No.: 9)), HvUBIrev (5′-ACA ACC AGA CAT GCT CCA ACC T-3′ (Seq ID No.: 10)). The GenBank accession numbers are as previously mentioned for Nicotiana benthamiana (NtPDS) and barley (HvPDS) PDS cDNA and are U66264 and X04133 for Nicotiana benthamiana (NtUBI) and barley (HvUBI) ubiquitin cDNA respectively. Primer concentrations giving the lowest threshold cycle (Ct) value were selected for further analysis. Detection of real-time PCR products was done by the incorporation of the fluorescent DNA dye SYBR green using the QuantiTect™ SYBR® Green PCR kit (Qiagen Ltd) following manufacturer's recommendations in an ABI PRISM 7700 Sequence Detection System (Applied Biosystems, USA) using 10-fold dilution in sterile water of first strand cDNA reaction mixes. All reactions were heated to 95° C. for 15 min, followed by 40 cycles of 94° C. for 15 sec, 60° C. for 30 sec and 72° C. for 30 sec. All calculations and statistical analysis were performed as described in the ABI PRISM 7700 Sequence Detection System User Bulletin #2 (Applied Biosystems, USA).

Plant Material and Growth Conditions

All work involving virus-infected material was carried out in containment glasshouses under SEERAD licences GM/141/2002, GM/160/2002 and GM/162/2002. Transgenic N. benthamiana line 16c carrying the 35S::GFP::tmos transgene have been previously described (Ruiz et al., 1998). GFP fluorescence was monitored as described previously (Lacomme and Santa Cruz, 1999). Plants infected with TMV constructs were maintained in a Snijder Climatic Cabinet (Snijder, Tilburg, Holland) with a photoperiod of 16 hours. Light intensity was approximately 400 μmol.m⁻².s⁻¹ and at either 32° C. or 24° C. for infectivity tests on Nicotiana edwardsonii. Barley cultivar Black Hulless plants infected with BSMV were kept in controlled environment chambers with a 16-hr photoperiod (22° C., light intensity ranging from 400 μmol.m⁻².s⁻¹ to 1000 μmol.m⁻².s⁻¹).

Plant Inoculations

Generation of infectious TMV and BSMV RNAs from cDNA clones and inoculation procedures were as described previously (Toth et al., 2002; Holzberg et al. 2002). Infectivity tests of TMV constructs were performed on Nicotiana glutinosa or Nicotiana edwardsonii (NN) as described by Toth et al. (2002).

TABLE 1
Real time PCR determination of normalised relative amount
PDS mRNA levels in silenced and control N. benthamiana plants
challenged with TMV constructs.

| | | Relative PDS mRNA |
|---|---|---|
| | Amount normalised | levels^{c} |
| Constructs | PDS mRNA^{a} (A.U.^{b}) | (%) |
| TMV | 1.244 | 100 |
| TMV.PDSas | 0.424 | 34 |
| TMV.hpPDS₄₀ | 0.251 | 20 |
| TMV.hpPDS₆₀ | 0.159 | 13 |

^{a}Value represents the mean from three plants per construct from two independent experiments carried out in triplicate. Least significant difference of means at 5% level = 0.163.
^{b}Arbitrary Unit.
^{c}Related to TMV control

TABLE 2
Real time PCR determination of normalised relative amount
PDS mRNA levels in silenced and control barley plants challenged
by BSMV constructs.

| | | Relative PDS mRNA |
|---|---|---|
| | Amount normalised | levels^{c} |
| Constructs | PDS mRNA^{a} (A.U.^{b}) | (%) |
| BSMV.GFP | 1.397 | 100 |
| BSMV.PDSs | 0.356 | 26 |
| BSMV.PDSas | 0.198 | 14 |
| BSMV.hpPDS_{Hv60} | 0.143 | 10 |
| BSMV.hpPDS_{Nb60} | 1.048 | 75 |

^{a}Value represents the mean from three plants per construct carried out in triplicate. Least significant difference of means at 5% level = 0.55.
^{b}Arbitrary Unit.
^{c}Related to BSMV.GFP control

References

Baulcombe, D. C. (1999) Fast forward genetics based on virus-induced gene silencing. Curr. Opin. Plant Biol. 2, 109-113.

Baulcombe, D. C. (2001) Diced defence. Nature, 409, 295-296.

Boutla, A., Kalantidis, K., Tavernarakis, N., Tsagris, M. and Tabler, M. (2002) Induction of RNA interference in Caenorhabditis elegans by RNAs derived from plants exhibiting post-transcriptional gene silencing. Nucleic Acids Research, 30, 1688-1694.

Buck, K. W. (1999) Replication of tobacco mosaic virus RNA. Phil. Trans. R. Soc. Lond. 354, 613-627.

Chuang, C. F. and Meyerowitz, E. M. (2000) Specific and heritable genetic interference by double-stranded RNA in Arabidopsis thaliana. Proc. Natl. Acad. Sci. USA, 97, 4985-4990.

Cogoni, C. and Macino, G. (2000) Post-transcriptional gene silencing across kingdoms. Curr. Opin. Gen. Devel., 10, 638-643.

Dalmay, T., Hamilton, A., Ruud, S., Angell, S. and Baulcombe D.C. (2000) An RNA-dependant RNA polymerase gene in Arabidopsis is required for post-transcriptional gene silencing mediated by a transgene but not by a virus. Cell, 101, 543-553.

De Buck, S., Van Montagu, M. and Depicker, A. (2001) Transgene silencing of invertedly repeated transgenes is released upon deletion of one of the transgenes involved. Plant Mol. Biol. 46, 433-445.

Eckes, P., Roshal, S., Schell, J. and Willmitzer, L. (1986) Isolation and characterization of a light-inducible, organ-specific gene from potato and analysis of its expression after tagging and transfer into tobacco and potato shoots. Mol. Gen. Genet. 205, 14-22.

Fagart, M. and Vaucheret, H. (2000) (Trans)gene silencing in plants: How many mechanisms? Ann. Rev. Plant Physiol. Plant Mol. Biol. 51, 167-194.

Haseloff, J., Siemering, K. R., Prasher, D. C. and Hodge, S. (1997) Removal of a cryptic intron and subcellular localization of green fluorescent protein are required to mark transgenic Arabidopsis plants brightly. Proc. Natl. Acad. Sci. USA, 94, 2122-2127.

Hamilton, A., Brown, S., Han, Y., Ishizuka, M., Lowe, A., Alpuche-solis, G. A. and Grierson, D. (1998) A transgene with repeated DNA causes high frequency, post-transcriptional suppression of ACC-oxidase gene expression in tomato. Plant J. 15, 737-746.

Holzberg, S., Brosio, P., Gross, C. and Pogue, G. P. (2002) Barley stripe mosaic virus-induced gene silencing in a monocot plant. Plant J. 30, 315-327.

Ibrahim, A. F. M., Watters, J. A., Clark, G. P., Thomas, C. J. R., Brown, J. W. S. and Simpson, C. G. (2001) Expression of intron-containing GUS constructs is reduced due to activation of a cryptic 5′ splice site. Mol. Genet. Genomics 265, 455-460.

Johansen, L. K. and Carrington, J. C. (2001). Silencing on the spot. Induction and suppression of RNA silencing in the Agrobacterium-mediated transient expression system. Plant Physiol. 126, 930-938.

Kielland-Brandt, M. C. (1974) Studies on the biosynthesis of tobacco mosaic virus. VII. Radioactivity of plus and minus strands in different forms of viral RNA after labelling of tobacco leaves. J. Mol. Biol. 87, 489-503.

Kumagai, M. H., Donson, J., della-Cioppa, G., Harvey, D., Hanley, K. and Grill, L. K. (1995) Cytoplasmic inhibition of carotenoid biosynthesis with virus-derived RNA. Proc. Natl. Acad. Sci. USA, 92, 1679-1683.

Lacomme, C. and Santa Cruz, S. (1999) Bax-induced cell death in tobacco is similar to the hypersensitive response. Proc. Natl. Acad. Sci. USA, 96, 7956-7961.

Lacomme, C., Pogue, G. P., Wilson, T. M. A. and Santa Cruz, S. (2001) Plant viruses. In Genetically Engineered Viruses: Development and applications, (Ring, C. J. A. and Blair. E. D., eds). Oxford, UK: BIOS. Scientific Publishers pp. 59-105.

Levin, J. Z., de Framond, A. J., Tuttle, A., Bauer, M. W. and Heifetz, P. B. (2000) Methods of double-stranded RNA-mediated gene inactivation in Arabidopsis and their use to define an essential gene in methionine biosynthesis. Plant Mol. Biol. 44, 759-775.

Mallory, A. C., Ely, L., Smith, T. H., Marathe, R., Anandalakshmi, R., Fagart, M., Vaucheret, H., Pruss, G., Bowman, L. and Vance, V. B. (2001) Hc-Pro suppression of transgene silencing eliminates the small RNAs but not transgene methylation or the mobile signal. Plant Cell, 13, 571-583.

Muskens, M. W. M., Vissers, A. P. A., Bol, J. N. M. and Kooter, J. M. (2000) Role of inverted DNA repeats in transcriptional and post-transcriptional gene silencing. Plant Mol. Biol. 43, 243-260.

Nagy, P. D. and Simon, A. E. (1997). New insights into the mechanisms of RNA recombination. Virology, 235, 1-9.

Rateliff, F., Martin-Hernandez, A. M. and Baulcombe, D. C. (2001) Tobacco rattle virus as a vector for analysis gene function by silencing. Plant J. 25, 237-245.

Ruiz, M. T., Voinnet, O. and Baulcombe, D. (1998) Initiation and maintenance of virus-induced gene silencing. Plant Cell, 10, 937-946.

Schweizer, P., Pokorny, J., Schulze-Lefert, P. and Dudler, R. (2000) Double-stranded RNA interferes with gene function at the single-cell level in cereals. Plant J. 24, 895-903.

Shivprasad, S., Pogue, G. P., Lewandowski, D. J., Hidalgo, J., Donson, J., Grill, L. K. and Dawson, W. O. (1999) Heterologous sequences greatly affect foreign gene expression in tobacco mosaic virus-based vectors. Virology, 255, 312-323.

Smith, N. A., Singh, S. P., Wang, M. B., Stoutjesdijk, P. A., Green, A. G. and Waterhouse, P. M. (2000) Total silencing by intron-spliced hairpin RNAs. Nature, 407, 319-320.

Simon, A. E. and Bujarski, J. J. (1997) RNA-RNA recombination and evolution in virus-infected plants. Annu. Rev. Phytopathol. 32, 337-362.

Tenllado, F. and Diaz-Ruiz, J. R. (2001) Double-stranded RNA-mediated interference with plant virus infection. J. of Virology, 75, 12288-12297.

Thomas, C. L., Jones, L., Baulcombe, D. C. and Maule, A. (2001) Size constraints for targeting post-transcriptional gene silencing and for RNA-mediated methylation in Nicotiana benthamiana using a potato virus X vector. Plant J. 25, 417-425.

Toth, R., Pogue, G. P. and Chapman, S. (2002) Improvement of the movement and host range properties of a plant virus vector through DNA shuffling. Plant J. 30, 593-600.

Varsha Wesley, S. et al,, Waterhouse P. M. (2001) Construct design for efficient, effective and high-throughput gene silencing in plants. Plant J. 27, 581-590.

Voinnet, O., Pinto, Y. M. and Baulcombe D. C. (1999) Suppression of gene silencing: a general strategy used by diverse DNA and RNA viruses. Proc. Natl. Acad. Sci. USA, 96, 14147-14152.

Voinnet, O., Lederer, C. and Baulcombe, D. C. (2000) A viral movement protein prevents spread of the gene silencing signal in Nicotiana benthamiana, Cell, 103, 157-167.

Voinnet, O. (2001) RNA silencing as a plant immune system against viruses. Trends in Genetics, 17, 449-458.

Waterhouse, P. M., Graham, M. W. and Wang, M. B. (1998) Virus resistance and gene silencing in plants can be induced by simultaneous expression of sense and antisense RNA. Proc. Natl. Acad. Sci. USA, 95, 13959-13964.

All of the above references are incorporated herein by reference in their entirety.

What is claimed is:

1. A DNA construct containing an infectious clone of an RNA virus containing a hairpin sequence corresponding to a gene encoded within the nucleus of the target host, said hairpin sequence being a sequence fragment of greater than 20 bp in length where the sequence fragment in the sense orientation is followed by different sequence fragment, derived from the first in the reverse complement orientation, with: no intervening sequence; or an intervening sequence of no greater than the collective length of the two sequence fragments comprising the hairpin.

2. A cytoplasmic inhibition of nuclear gene expression resulting from hairpin RNA expression from an RNA virus genome in accordance with claim 1.

3. A plant host experiencing cytoplasmic inhibition of gene expression following infection with an RNA virus genome containing a hairpin nucleotide sequence in accordance with claim 1.

4. An animal host experiencing cytoplasmic inhibition of gene expression following infection with an RNA virus genome containing a hairpin nucleotide sequence in accordance with claim 1.

5. A method for determining nuclear gene function through a process of cytoplasmic inhibition of gene expression following infection with an RNA virus genome containing a hairpin nucleotide sequence in accordance with claim 1.

6. A hairpin sequence vector in accordance with claim 1 comprising a tobacco mosaic virus.

7. A hairpin sequence vector in accordance with claim 1 comprising a barley striped mosaic virus genome.

8. A virus vector for cytoplasmic gene silencing applications, in accordance with claim 1, comprising a very short, yet highly active gene silencing inducer, such as said hairpin sequence, wherein said virus vector exhibits improved genetic stability.

9. A vector as specified in claim 1, that contains a 20-30 nucleotide hairpin sequence for infection of mammalian cells and delivery of hairpin RNA to the cytosol for cytoplasmic gene inhibition.

10. A vector as specified in claim 9, derived from the alphavirus, rubivirus virus families.


SEQUENCE LISTING (12 sequences)

SEQ ID NO: 1 - DNA, 29 bp, Artificial Sequence
  Primer
taaattaattaattcctgatcgagtcaac

SEQ ID NO: 2 - DNA, 24 bp, Artificial Sequence
  Primer
atttgaattctttatgaaattgag

SEQ ID NO: 3 - DNA, 24 bp, Artificial Sequence
  Primer
ttcttcaggagaaacatggttcaa

SEQ ID NO: 4 - DNA, 18 bp, Artificial Sequence
  Primer
tccacaatcggcatgcaa

SEQ ID NO: 5 - DNA, 21 bp, Artificial Sequence
  Primer
tccaggacaaggagggtatcc

SEQ ID NO: 6 - DNA, 22 bp, Artificial Sequence
  Primer
tagtcagccaaggtccttccat

SEQ ID NO: 7 - DNA, 24 bp, Artificial Sequence
  Primer
tggagcttatcccaatgtacagaa

SEQ ID NO: 8 - DNA, 21 bp, Artificial Sequence
  Primer
tgtattcccctggcttgtttg

SEQ ID NO: 9 - DNA, 22 bp, Artificial Sequence
  Primer
gcaagtaagtgcctggtcatga

SEQ ID NO: 10 - DNA, 22 bp, Artificial Sequence
  Primer
acaaccagacatgctccaacct

SEQ ID NO: 11 - DNA, 60 bp, Artificial Sequence
  First of two sequences depicted In FIG. 4B
caaaaatattgaagtatcatgttgtcaaaaccccaaggtctgtttataaaactgtgccag

SEQ ID NO: 12 - DNA, 60 bp, Artificial Sequence
  Second of two sequences depicted in FIG. 4B
cgaagattcttaaataccatgttgtgaagacaccaaggtctgtttacaagaccgtcccga